(19) United States
(12) Patent Application Publication
(10) Pub. No.: US 2015/0327870 A1
(43) Pub. Date: Nov. 19, 2015

(54) METHODS, SYSTEMS, AND DEVICES FOR APPLYING TARGET FORCE TO A RADIAL ACCESS PUNCTURE SITE

(71) Applicant: Abbott Cardiovascular Systems, Inc., Santa Clara, CA (US)

(72) Inventors: Aaron M. Fortson, Fremont, CA (US); Douglas H. Mehl, Redwood City, CA (US); Timothy C. Reynolds, Sunnyvale, CA (US)

(73) Assignee: Abbott Cardiovascular Systems, Inc., Santa Clara, CA (US)

(21) Appl. No.: 14/278,945

(22) Filed: May 15, 2014

(51) Int. Cl. A61B 17/135; A61B 17/132

(52) U.S. Cl. CPC A61B 17/135; A61B 17/1325

(57) ABSTRACT

Systems and methods for applying targeted force to a radial access puncture site to provide hemostasis and close the puncture site. The system may include a cuff for placement over a wrist, a fluid source from which an inflation fluid may be delivered into an inflatable balloon associated with the cuff during use, and an inflatable balloon attached to the cuff. The balloon is disposed over the puncture site and inflated during use. The system may include one or more features configured to focus or target the applied force to the puncture site. For example, the balloon may include varying wall thickness, with a thinner-walled portion that applies a focused pressure to the puncture site, two balloons where one applies focused force, or an outer sheath with a hole placed over the balloon so that a portion of the balloon protrudes through the hole and applies focused force.

### BACKGROUND

The present invention relates generally to apparatus and methods for the closing of an access passage opened within a body lumen. More particularly, the present invention relates to techniques for closure of arterial and venous puncture sites, particularly, a radial artery access site.

A number of diagnostic and interventional vascular procedures are now performed translumenally. A catheter is introduced into the vascular system at a convenient access location and guided through the vascular system to a target location using established techniques. Such procedures require vascular access, which is usually established during the well-known Seldinger technique. Vascular access is generally provided through an introducer sheath, which is positioned to extend from outside the patient body into the vascular lumen.

Accessing the vasculature through the radial artery, rather than the larger femoral artery has become increasingly popular as the structures employed in such procedures and introduced into the vasculature become smaller. No matter the access site, when vascular access is no longer required, the introducer sheath is removed and bleeding at the puncture site stopped. Various techniques are currently available for providing hemostasis (the cessation of bleeding).

One approach is to use fasteners, suturing, etc. to close the site. Such techniques can be problematic due to the need to target suture capture for retrieval through the artery or other body lumen wall, which often requires complicated mechanisms. In addition, where the site is a radial artery access site, such fastening or closure devices are typically designed for use in larger access sites, such as a femoral access site, and may thus represent “overkill” in closing a radial access site. In addition, such techniques demand a high level of practitioner skill. Finally, even when performed properly, such procedures may still result in minor oozing from the access tract adjacent the site.

Another approach is to apply external force near and upstream from the puncture site, typically by manual or “digital” compression. This approach is time consuming, frequently requiring one-half hour or more of practitioner compression before hemostasis is assured. Additionally, such compression techniques rely on clot formation, which can be delayed until anticoagulants used in vascular therapy procedures (such as for heart attacks, stent deployment, non-optical PTCA results, and the like) wear off. This can take two to four hours, thereby increasing the time required before completion of a typical compression technique. The compression procedure is further often uncomfortable for the patient, frequently requiring analgesics to be tolerable. Moreover, the application of excessive pressure can at times totally occlude the underlying blood vessel, resulting in ischemia thrombosis, and/or nerve damage.

Thus, compression techniques often require significant time of highly trained individuals, while dedicating such personnel to this task is both expensive and inefficient. As such, there continues to be a need for devices and methods that might provide improved closure of radial access sites.

### BRIEF SUMMARY

The present disclosure describes devices, systems and methods for applying targeted compressive force to a radial access puncture site to provide hemostasis and closure of the site. The device may comprise a cuff (e.g., a band) for placement over (e.g., around) a wrist of a patient, and an inflatable balloon attached to the cuff and disposed over the radial access puncture site during use. The device may include one or more features that provide for focused, targeted pressure over the puncture site. For example, in an embodiment, the balloon may include a wall thickness that varies so as to include a relatively thicker wall portion and a relatively thinner wall portion so that the thinner wall portion extends further from the cuff than the thicker wall portion under fluid pressure from the fluid source. The thinner wall portion is positioned over the radial access puncture site, so that the extended thinner wall portion of the balloon applies a focused pressure to the radial access puncture site. A related system may include such a cuff device, and a fluid source from which an inflating fluid may be delivered into the inflatable balloon associated with the cuff during use.

Application of such a focused pressure may provide for faster, more efficient hemostasis at the puncture site. Because the focused pressure is applied by the balloon and cuff device of such a system, continuous attendance of a highly trained medical practitioner may not be required.

Another embodiment of a device may include a cuff for placement over a wrist of a patient, an inflatable balloon attached to the cuff and disposed over the radial access puncture site during use, and an outer sheath positionable over the inflatable balloon during use. The outer sheath may include a hole through which a portion of the inflated balloon may be received, the portion of the inflated balloon protruding through the hole of the sheath during use such that the protruding portion of the balloon applies a focused pressure to the radial access puncture site during use.

Another embodiment of a device for applying targeted force to a radial access puncture site may include a cuff for placement over a wrist of a patient and an inflatable balloon structure attached to the cuff and disposed over the radial access puncture site during use. The inflatable balloon structure includes a first relatively smaller balloon disposed within a second relatively larger balloon. The first balloon is disposed over the radial access puncture site, and extends beyond (e.g., above) an outer surface of the second balloon during use, so that the first balloon applies a focused pressure to the radial access puncture site during use that is greater than pressure applied by the second balloon to areas surrounding the puncture site.

Another embodiment of the present disclosure is directed to a method for applying targeted force to a radial access puncture site. The method may include placing a cuff of a device for applying targeted force over a wrist of a patient. The device may include the cuff for placement over a wrist of a patient, and an inflatable balloon structure attached to the cuff that is disposed over the radial access puncture site during use so that the inflatable balloon structure applies pressure to the radial access puncture site during use. The system includes one or more features that allow for application of a focused pressure to the radial access puncture site during use. Any of the above described structures may be employed for applying the focused pressure to the radial access puncture site.

For example, in an embodiment, the inflatable balloon structure may comprise an inflatable balloon having a wall thickness that varies so as to include a relatively thicker wall portion, and a relatively thinner wall portion. The thinner wall portion extends further from the cuff than the thicker wall portion under fluid pressure from a fluid source inflating the balloon. The thinner wall portion is positioned over the radial access puncture site, so that the extended thinner wall portion applies a focused pressure to the puncture site. In another example, the inflatable balloon structure includes an inflatable balloon and an outer sheath positionable over the balloon during use. The sheath includes a hole into which a portion of the inflatable balloon is received. The portion of the inflated balloon protrudes through the hole of the sheath during use, so that the protruding portion applies a focused pressure to the puncture site during use. In another example, the inflatable balloon structure includes two balloons—a first smaller balloon disposed within a second larger balloon. The first balloon is disposed over the puncture site and extends beyond (e.g., above) an outer surface of the second balloon during use. As a result, the first balloon applies a focused pressure to the puncture site that is greater than pressure applied by the surrounding second balloon, which may apply pressure to areas surrounding the puncture site.

The structures of the cuff device may be adjusted in position (e.g., the cuff, the balloon structure, a rigid or semi-rigid member, etc.) to align the specific feature (e.g., thinned balloon portion, hole of the sheath, or the first, smaller balloon) that provides application of focused pressure over the puncture site. The inflatable balloon structure may be inflated, providing a focused pressure to the puncture site to provide hemostasis to the puncture site while limiting risk of damage to nerves adjacent the puncture site.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. Embodiments of the invention will be described and explained with additional specificity and detail through the use of the accompanying drawings in which:

FIGS. 1A-1B are schematic perspective views of an exemplary system including a cuff device for placement over a radial access puncture site, with FIG. 1A showing the puncture site and cuff before the cuff is placed over the puncture site, and FIG. 1B showing the cuff device placed over the puncture site;

FIG. 1C is a schematic view of another exemplary cuff device;

FIGS. 1D and 1E are schematic perspective views of yet another cuff device, with FIG. 1D showing the cuff device before placement over the puncture site, and FIG. 1E showing the cuff device placed over the puncture site;

FIG. 2A is a close up view showing the inflatable balloon and surrounding portions of the cuff device of FIGS. 1A-1B;

FIG. 2B is a cross-sectional view of the cuff device of FIG. 2A, ready for positioning over a radial access puncture site, with the balloon of the cuff device inflated to apply pressure against the puncture site;

FIG. 2C is a cross-sectional view similar to that of FIG. 2B, with the balloon not fully inflated, so as to not include the bumped protruding portion as in FIG. 2B, and showing an alternative configuration for attaching the balloon, the rigid or semi-rigid backing member, and cuff;

FIG. 2D is a cross-sectional view similar to that of FIG. 2B, with the balloon not fully inflated, so as to not include the bumped protruding portion as in FIG. 2B;

FIG. 3A is a schematic top or plan view of an exemplary configuration for the balloon of the cuff device;

FIG. 3B is an schematic top or plan view of another exemplary configuration for a balloon of the cuff device;

FIG. 3C is an schematic top or plan view of yet another exemplary configuration for a balloon of the cuff device;

FIG. 4A is a perspective view of an alternative system including a cuff device and an outer sheath having a hole through which a portion of the inflated balloon may protrude when the sheath is applied over the balloon;

FIG. 4B is a cross-sectional view of the cuff device and outer sheath of FIG. 4A, showing the balloon inflated, with a portion of the balloon protruding through the hole of the outer sheath positioned over the balloon;

FIG. 5A is a perspective view of an alternative system including first and second balloons, the first balloon disposed within the second balloon; and

FIG. 5B is a cross-sectional view of the system of FIG. 5A.

### DETAILED DESCRIPTION

### I. Introduction

In one aspect, the present disclosure describes methods, systems, and devices included as part of such systems for applying targeted force to a radial access puncture site to arrest bleeding at the puncture site. Such a device may include a cuff (e.g., band) for placement over a wrist of a patient, and an inflatable balloon attached to the cuff and disposed over the radial access puncture site. The device or system includes one or more features that provide for targeted or focused application of the force to the puncture site. For example, in an embodiment, the balloon may have a varying wall thickness, so as to include a relatively thicker wall portion and a relatively thinner wall portion, so that when the balloon is inflated, the thinner wall portion extends further from the cuff than the thicker wall portion. Placement of the thinner wall portion over the puncture site allows this extended thin-walled portion of the balloon to apply a targeted pressure to the puncture site that is greater than pressure that may be applied to areas surrounding the site (i.e., applied by the thicker-walled portion). An associated system may include a cuff device, and a fluid source from which an inflation fluid may be delivered into the inflatable balloon during use.

In another embodiment, the device or system includes an outer sheath that may be positionable over the balloon during use. The sheath may include a hole formed therein, so that when positioned over the balloon, a portion of the balloon is received into the hole, and protrudes through the hole when the balloon is inflated. The protruding portion of the balloon may be positioned over the puncture site, so as to apply a targeted pressure to the puncture site that is greater than pressure applied to areas surrounding the site (i.e., applied by the portions of the balloon covered by the sheath).

In another embodiment, the inflatable balloon structure includes at least two balloons, with a relatively smaller balloon disposed within a relatively larger balloon. The first balloon may extend beyond an outer surface of the second balloon, so that when the first balloon is positioned over the puncture site, a targeted pressure is applied by the first balloon to the puncture site that is greater than pressure applied by the surrounding second balloon (i.e., applied to areas surrounding the puncture site). In each of these embodiments, the greatest pressure may be applied over the puncture site, while decreased pressure is applied (if at all) to locations surrounding the puncture site. This may advantageously provide sufficient pressure for hemostasis to be achieved at the puncture site, while limiting any risk of damage to nerve tissue adjacent the puncture site.

### II. Exemplary Devices and Methods

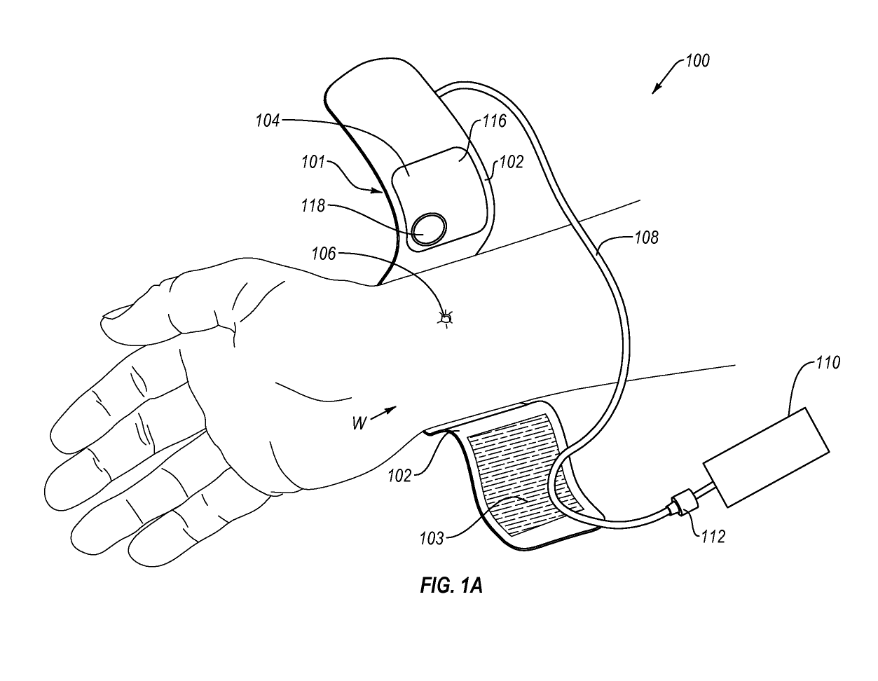

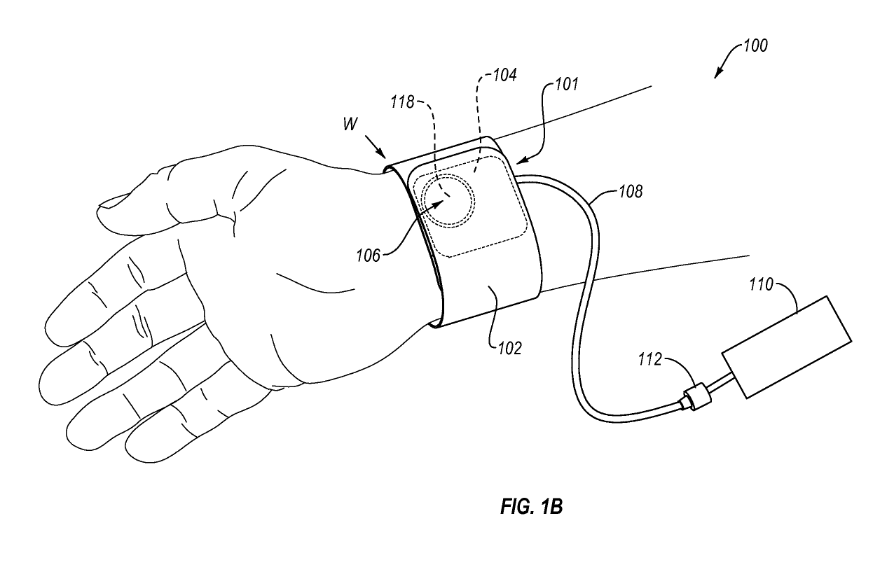

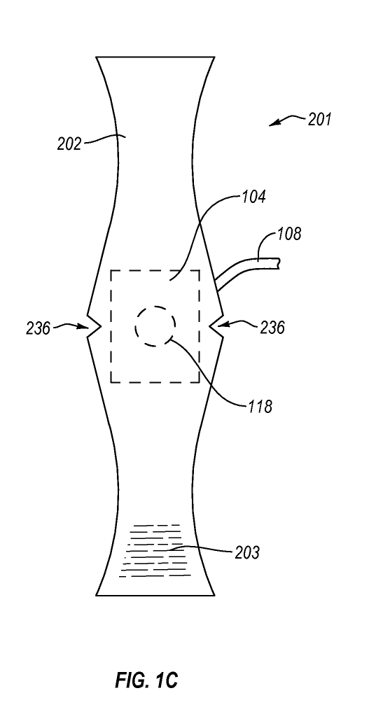

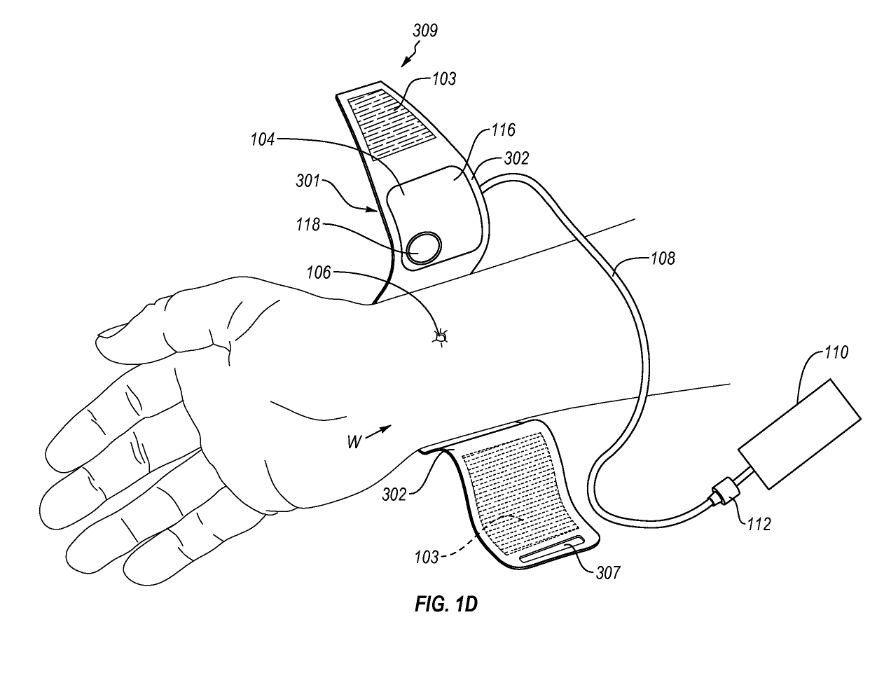

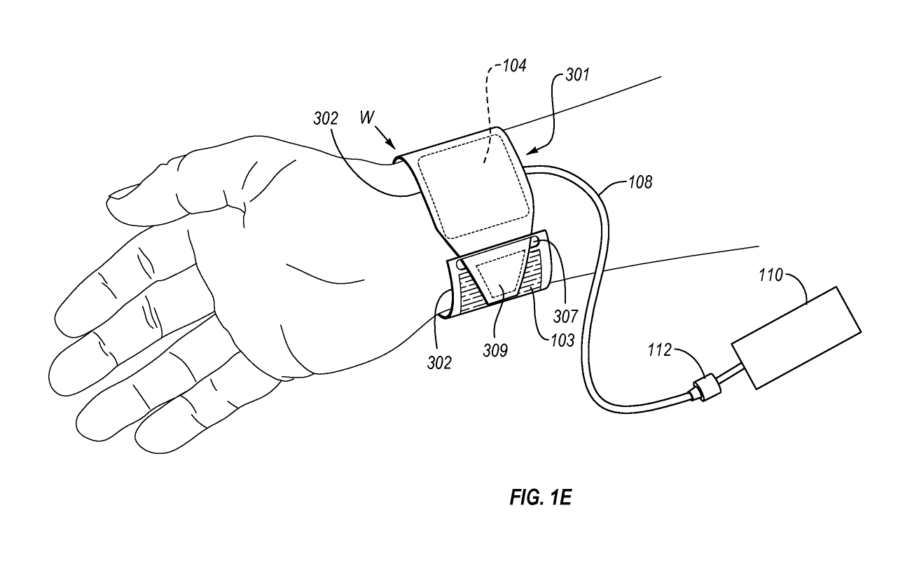

Referring to FIGS. 1A-1B, an exemplary system 100 including device 101 is shown, including a cuff 102 that can be placed over the wrist W of a patient. Cuff 102 may be configured as any suitable structure that can be disposed over (e.g., around) a wrist of a patient. For example, cuff 102 may comprise an elongate band, as seen in FIG. 1A that may be wrapped over and secured to itself (e.g., similar to a blood pressure cuff). For example, cuff 102 may include any suitable fastener for securing the cuff 102 around the patient's wrist, such as snaps, hook and loop fasteners (e.g., VELCRO), a buckle, a self-adhesive (e.g., a pressure sensitive adhesive including a cover layer that is peeled off immediately prior to placement), etc. FIG. 1A illustrates one part of hook and loop fastener (e.g., hooks 103) on one face of band 102, while the other portion of the hook and loop mechanism (e.g., loops) may be disposed on a corresponding face of another portion of band 102. Of course, other fastening mechanisms may alternatively be employed.

In another embodiment, the cuff 102 may be configured as a continuous round band (e.g., generally circular or other rounded shape) that is received over the wrist. For example, the cuff 102 could comprise an elastic material that would allow it to be expanded as it is advanced over the hand, where it retracts to its original un-expanded configuration once over the wrist. Such a continuous round band could be provided by attaching opposed ends of an elongate band (e.g., as seen in FIG. 1A) to one another using a suitable fastening mechanism (e.g., stitched together, VELCRO, self-adhesive, etc.). FIG. 1A shows radial access puncture site 106, just as cuff 102 is being prepared for placement thereover. The inflatable balloon 104 may be disposed on cuff 102 so as to be positioned over puncture site 106 as cuff 102 is tightened around wrist W. In particular, a thinner-walled portion 118 of balloon 104 or other feature for applying focused pressure from balloon 104 may be disposed on cuff 102 so as to be directly over puncture site 106. FIG. 1B illustrates band 102 once it has been wrapped around the wrist W, with balloon 104 (e.g., and thinner-walled portion 118) positioned over puncture site 106, so as to apply targeted pressure thereto.

Inflatable balloon structure 104 may be attached to cuff 102, where balloon 104 is positioned over the radial access puncture site 106 (e.g., somewhat above the center of the wrist W on the inside of the arm), as seen in FIGS. 1A-1B. FIG. 1B shows balloon 104 positioned over radial artery puncture site 106 of the right arm. It will be apparent that a similar cuff and balloon could be positioned over a radial artery puncture site on the left arm. Inflation of the balloon structure 104 causes application of a pressure that is targeted to the puncture site 106, so as to compress site 106 and lead to hemostasis, closing the puncture site 106. In addition to cuff 102 and balloon 104, system 100 may further include a fluid delivery line 108, a fluid source 110 (shown schematically), and a valve 112 which may be in fluid communication (e.g., selective fluid communication) with balloon 104. An inflation fluid (e.g., air, saline, water, etc.) may be delivered from source 110, through valve 112, through line 108, and into balloon 104 of device 101, inflating balloon 104, which inflation fluid may exert the desired targeted pressure to puncture site 106. Fluid source 110 may comprise a syringe or other suitable structure for containing and/or compressing the fluid.

Balloon 104 may be any suitable inflation structure, e.g., that can be inflated upon introduction of an inflation fluid. Inflation of balloon 104 may be achieved using fluid source 110 (e.g., a syringe or other suitable source). In an embodiment, inflation fluid may be delivered through line 108 to balloon 104. In another embodiment, a syringe or other fluid source may be coupled to balloon 104 directly, without an intervening fluid delivery line 108. In an embodiment, a liquid inflation fluid (e.g., water, saline, etc.) is employed, which may provide a greater targeted pressure to puncture site 106 than a gaseous fluid (e.g., air, nitrogen, etc.).

While in an embodiment balloon 104 may be pre-inflated prior to placement (e.g., during manufacture or immediately prior to placement), the ability to inflate balloon 104 may be advantageous as a practitioner may position cuff 102 and balloon 104 as desired (e.g., with a desired portion of balloon 104 over puncture site 106), and then inflate balloon 104, to provide a greater level of applied compressive pressure than may be readily achieved were balloon 104 pre-inflated. Such a pre-inflated balloon may not need to be attachable to a fluid source 110 (e.g., it may be permanently sealed in an inflated configuration during manufacture).

In an embodiment, the inflation fluid may be cooled below ambient temperature, to provide cooling to puncture site 106, e.g., to limit inflammation and/or accelerate hemostasis at the site. In another embodiment, cooling may be provided by passing a cooling fluid separate from the inflation fluid through channels formed within the inflation balloon, cuff, an optional rigid or semi-rigid member that backs the balloon, etc. Such cooling channels may be configured to provide separation between the inflation fluid and any cooling fluid. An ice pack or other cooling pack could also be included within the cuff device (e.g., a compartment in the cuff device that receives and retains an ice or cooling pack). In other embodiments, heating could alternatively be provided through similar mechanisms (e.g., heating the inflation fluid, a separate heating fluid, a heating pack, etc.), where heating may be desirable.

In addition, where desired, a hemostatic or coagulating agent may be applied to the puncture site and/or the balloon 104 (e.g., to that portion of balloon 104 that may contact puncture site 106). Examples of such hemostatic and coagulating agents include, but are not limited to aluminum compounds, iron-based compounds, and combinations thereof. Specific examples of such include, but are not limited to potassium aluminum sulfate, aluminum ammonium sulfate, aluminum sulfate, aluminum chlorohydrate, aluminum acetate, aluminum chloride, ferric sulfate, ferric subsulfate, ferric chloride, and mixtures thereof. Other such agents may include permanganates, tannins, zinc chloride, chitosan, etc. In one embodiment a vasoconstrictor such as epinephrine and/or propylhexedrine may also be provided. Such agents may be coated on the balloon, or delivered through pores in an outer layer of the balloon, etc. for delivery to puncture site 106.

In an embodiment, system 100 or device 101 may be provided in a single sterilized kit (e.g., a package including the cuff, attached balloon, fluid delivery lines, fluid source, etc.) In another embodiment, the cuff and balloon device 101 may be employed with existing tubing, syringes, other fluid sources, etc. commonly found in a catheterization lab, hospital, or similar clinical facility.

FIGS. 1C-1E illustrate additional strap closure embodiments for exemplary cuff devices. For example, FIG. 1C schematically shows an exemplary cuff device 201 configured for placement over wrist W. Cuff 202 may be configured as an elongate band, similar to cuff 102 shown in FIGS. 1A-1B, but is shown as including alignment notches 236 on one or both peripheral edges of the elongate cuff 102, so that the vertex of notches 236 are aligned with one another, and with the portion of inflatable balloon 104 (e.g., a thin-walled portion 118) to be positioned over puncture site 106. Such notches 236 may aid the practitioner in correctly placing and positioning cuff 202, and particularly the desired portion of balloon 104 over the puncture site 106, even where balloon 104 (or the portion thereof to be placed over puncture site 106) may not be readily visible during placement. For example, it may be on the underside of the elongate strap material of cuff 202, being covered by fabric, a rigid or semi-rigid member, etc. Even with such covering, notches 236 disposed in the perimeter edge of cuff 202 may be advantageously visible, aiding the practitioner in proper placement and alignment. It will be appreciated that markings visible on the perimeter edge, rather than necessarily notches, may be employed for the same purpose. Of course, both may be provided (e.g., notches with marked or colored edges). One or both of respective ends of strap shaped cuff 202 may include self-adhesive 203, VELCRO, or other suitable mechanism for attachment of the opposed ends together.

FIGS. 1D-1E illustrate another cuff device embodiment 301 that may be similar to device 101 and cuff 102 shown in FIGS. 1A-1B, but is shown as including a slot 307 disposed in an end of strap or cuff 302, allowing an opposite end 309 to be inserted through slot 307, and cinched and secured (e.g., with VELCRO 103, or any other suitable mechanism) as seen in FIG. 1E. VELCRO 103 is shown on opposed surfaces of the strap, so as to be oriented to face one another once cuff 302 is disposed around wrist W. As shown, end 309 which is inserted through slot 307 may be tapered, so as to facilitate easier insertion into slot 307. Slot 307 may be formed in the cuff 302, and/or through an optional rigid or semi-rigid member between the cuff and balloon. It will be appreciated that a wide variety of cuff configurations will be suitable for use with the present invention. As such, it will be appreciated that any cuff device may include the features described below or elsewhere herein.

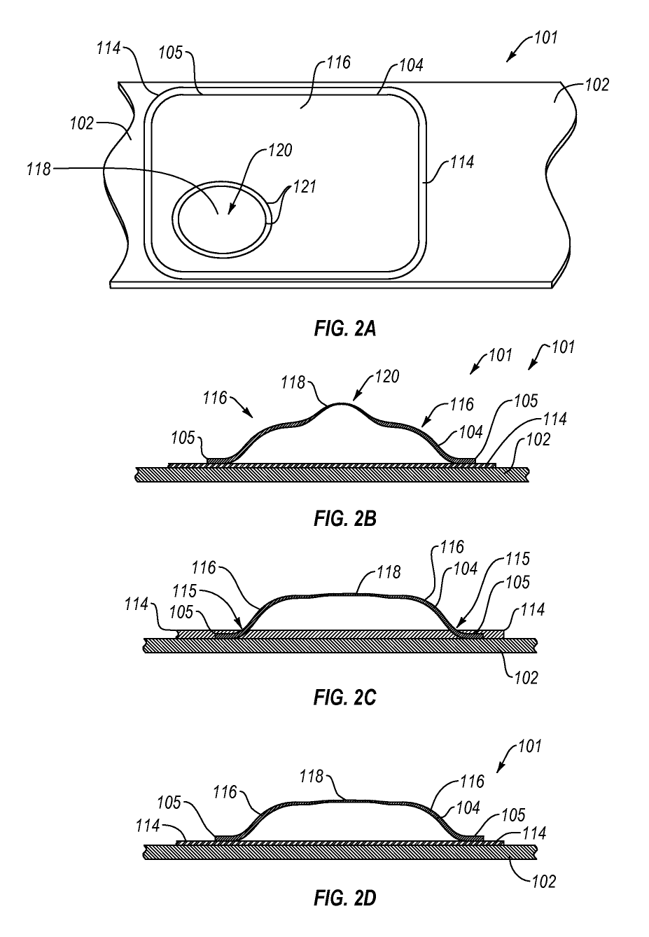

FIGS. 2A-2B illustrate close up and cross-sectional views, respectively, of a portion of an exemplary cuff device 101, which may be included in such a system. For example, as seen in FIGS. 2A-2B, system 100 and device 101 may include a rigid or semi-rigid (e.g., more rigid than balloon 104 and/or cuff 102) member 114 disposed between cuff 102 and balloon 104. Such a rigid or semi-rigid member may comprise a sheet of plastic, metal, paperboard, or other material that provides an increased degree of rigidity as compared to the balloon 104 (e.g., comprising a thin polymeric film) and/or cuff 102 (e.g., comprising a flexible fabric or similar material). Such a rigid or semi-rigid member 114 may serve to limit movement of balloon 104 in a direction opposite a direction of force applied to puncture site 106 (e.g., in a direction away from puncture site 106). For example, where provided, rigid member 114 may provide a structure which balloon 104 may “push against” to apply the desired force to puncture site 106. Member 114 may be localized (e.g., smaller than cuff 102 and/or balloon 104), or may match the configuration or surface area covered by balloon 104. For example, in the embodiment shown in FIGS. 2A-2B, rigid or semi-rigid member 114 may extend to or past the outer peripheral edge 105 of balloon 104. In another embodiment, the outer peripheral edge of member 114 may be disposed within the perimeter defined by balloon 104 (e.g., being localized to the puncture site 106 and immediate surrounding tissue). In the illustrated embodiment (e.g., see FIG. 2B), rigid or semi-rigid member 114 is disposed between cuff 102 and balloon 104, with balloon attached over member 114. In another embodiment, the peripheral edge 105 of balloon 104 may be sandwiched between cuff 102 and member 114 (e.g., through a perimeter slot 115 formed through member 114, as seen in FIG. 2C). Such a configuration may employ member 114 to aid in securing peripheral edge 105 of balloon in place.

In the embodiment illustrated in FIG. 2B, balloon 104 is shown attached indirectly to cuff 102, as balloon 104 is attached to rigid or semi-rigid member 114, which member 114 is in turn attached to cuff 102. Any suitable mechanism may be employed for such attachment. For example, one structure may be attached to another structure using stitching, adhesives, buttons, snaps, or any other suitable fastener. In an embodiment, rigid or semi-rigid member 114 may be stitched into cuff 102, and balloon 104 may be attached to member 114 using a suitable adhesive.

While shown with a rigid or semi-rigid member 114, it will be appreciated that in another embodiment no such member 114 may be present. For example, balloon 104 may be attached directly to cuff 102, without any member 114. Even where a rigid or semi-rigid member 114 is present, in an embodiment, at least a portion of balloon 104 may be directly attached to cuff 102, e.g., as seen in FIG. 2C, or where member 114 is smaller (i.e., smaller in footprint) than balloon 104, being localized to a very small area over puncture site 106, while balloon 104 may cover a larger area.

Balloon 104 may be particularly configured to apply a targeted compression force to puncture site 106, so as to apply greater force to puncture site 106 as compared to force that may be applied to regions that are adjacent to puncture site 106. For example, in accessing the radial artery through a radial access puncture site, a relatively small needle (e.g., about 0.03 inch in outside diameter) may be used, and introducer wires, catheters, sheaths, and other structures employed in forming the small puncture site and initiating access to the radial artery may be typically quite small (e.g., up to about 0.04 or 0.05 inch in outside diameter). A catheter sheath introduced into the radial artery may be somewhat larger, but still significantly smaller than those introduced into the femoral artery, as the radial artery is much smaller. For example, such a sheath may be up to about 6 Fr (about 2 mm, or about 0.08 inch) in outside diameter. Because the puncture site is typically relatively small in area, bleeding from puncture site 106 may be arrested through compression, and the patient may even be allowed to stand and walk soon after the procedure. This is in sharp contrast to closure after femoral access, where the patient may be required to remain still for several hours (e.g., at least about 5 hours). The system 100 and device 101 may be specifically configured to apply targeted pressure over the relatively small puncture site 106, and optionally a small area surrounding the puncture site, so as to achieve hemostasis and close puncture site 106, while limiting unneeded pressure so as to reduce risk of nerve damage or other unwanted complications or side-effects.

System 100 and device 101 may be configured to apply targeted pressure to puncture site 106 and a small adjacent area surrounding puncture site 106. For example, in an embodiment, system 100 and device 101 may apply targeted pressure to a region having an area of about 3 in² or less, about 2 in² or less, about 1 in² or less, about 0.75 in² or less, or about 0.5 in² or less. Applied pressure may be sufficient to provide hemostasis and closure of the puncture site, while not being so great, or applied over a large surface area as to cause nerve damage.

FIGS. 2A-2D illustrate an embodiment in which balloon 104 includes a varying wall thickness in order to apply a greater, targeted pressure at a given localized area, and lower pressure at another area (e.g., surrounding the target area). FIG. 2D shows balloon 104 at only an intermediate level of inflation, while FIG. 2B illustrates a higher level of inflation. As shown, balloon 104 may include a relatively thicker wall portion 116 and a relatively thinner wall portion 118. When inflated, thinner wall portion 118 may extend or protrude from cuff 102 a greater distance than adjacent thicker wall portion 116, as seen in FIG. 2B. With thinner wall portion 118 positioned over radial access site 106, the extending or protruding characteristic of “bubble” 120 applies a focused, targeted pressure to site 106, and the underlying radial artery. Such targeted compressive pressure allows hemostasis and closure of site 106 to occur. Because the pressure applied is localized (e.g., by bubble 120), a lower amount of pressure, if any significant pressure at all, is applied to regions surrounding the target area including site 106, by portion 116. Limiting the magnitude of pressure applied within this adjacent area may be beneficial, as it limits risk of nerve damage that may otherwise be associated with application of pressure over a larger area, away from puncture site 106. In an embodiment, at least a portion of the targeted pressure may be applied to the radial artery at a location “upstream” from puncture site 106.

The extended, protruding “bubble” portion 120 of balloon 104 may be positioned against puncture site 106, with cuff 102 and/or rigid or semi-rigid member 114 holding portion 120 firmly against site 106, as balloon 104 is attached to cuff 102, and cuff 102 is wrapped or otherwise placed over the wrist of the patient. Because of the targeted compressive force applied by bubble portion 120 to site 106, hemostasis may be quickly achieved, closing puncture site 106.

The transition between thicker-walled portion 116 and thinner walled portion 118 may be a gradual tapering of wall thickness, as seen in FIGS. 2B and 2D. In another embodiment, the thickness may include a step or abrupt change in thickness from portion 116 to portion 118.

Inflation of balloon 104 may be achieved by introducing a suitable inflation fluid from a fluid source 110 (e.g., a syringe or other source) directly into balloon 104, or through delivery line 108, inflating balloon 104 so as to cause thinner-walled portion 118 to protrude or extend further from cuff 102 relative to adjacent thicker walled portions 116, as shown in FIG. 2B. As a result, greater pressure may be applied by “bubble” portion 120, corresponding to thin-walled portion 118, as compared to thicker-walled portion 116. Positioning the thinner walled portion 120 over puncture site 106 causes greater pressure to be applied at that location than adjacent areas.

In an embodiment, thinner walled portion 118 may be differently colored as compared to coloring of adjacent thicker walled portions 116 of balloon 104. Such a configuration aids a practitioner in easily recognizing where thinner walled portion 118 is, particularly prior to inflation, before a “bubble” 120 may have formed. In another embodiment, other indicia may be included so as to mark and distinguish thinner walled portion 118 from adjacent thicker walled portions 116 (e.g., providing markings on portion 118, such as a perimeter outline 121, etc.).

The geometry of balloon 104, thinner walled portion 118 and thicker walled portion 116 may be as desired. FIGS. 3A-3C illustrate various exemplary, non-limiting embodiments. For example, in an embodiment as shown in FIG. 3A, balloon 104 may extend to the ends of cuff 102, and the thinner walled portion 118 of balloon 104 may be generally centrally disposed within balloon 104. As illustrated, in an embodiment, more than about 50% of the surface area of balloon 104 may be the thin-walled portion 118, and thin-walled portion 118 may be generally centrally disposed, within balloon 104 as a whole. As shown in FIG. 3A, the thin-walled portion 118 may extend to or nearly to edges 122 and 124 of balloon 104. Balloon 104 may extend to or nearly to edges 123 and 125 of cuff 102.

As shown in FIG. 3B, in another embodiment, thin-walled portion 118 may be located within balloon 104 offset from center, e.g., generally within one of four quadrants defined if balloon 104 were bisected in length and width. For example, thin-walled portion 118 may be disposed above a center axis of balloon 104, to the left (i.e., top left quadrant) of the other (e.g., width) bisecting axis. Positioning of thin-walled portion 118 within the overall geometry of balloon 104 may depend on where balloon 104 is attached to cuff 102. For example, FIG. 1A shows an alternative placement. In any case, the position of thin-walled portion may be such so as to correspond to the position of the radial access puncture site, as cuff 102 is placed over wrist W, as seen in FIGS. 1A-1B. The thin-walled portion 118 may represent only a relatively small fraction of the overall surface area of balloon 104 as a whole, so as to apply targeted force to a small target area at the puncture site 106. For example, the thin-walled portion 118 may be less than about 50%, less than about 40%, less than about 30%, less than about 25%, or less than about 20% of the surface area or “footprint” of balloon 104.

The geometric shape of thin-walled portion 118 may be of any desired shape. For example, FIG. 3A shows a generally rectangular shape, within a larger generally rectangular shaped balloon 104 as a whole. FIG. 3B illustrates a circular shaped thin-walled portion 118, similar to that of FIGS. 1A-1B and 2A. FIG. 3C illustrates an elongate rectangular shaped thin-walled portion 118. Various other shapes for the thin-walled portion 118, the thicker walled portion 116 (e.g., surrounding thin walled portion 118), and the balloon 104 as a whole may be employed (e.g., rectangular, square, circular, oval, other rounded shape, other closed curve shape, regular or irregular polygonal shaped, other closed geometric shape, etc).

In an embodiment, the balloon as a whole may have a size that is not larger than so as to cover the inside surface of the wrist (e.g., without wrapping around towards the front of the arm). For example, the balloon may cover only a small fraction of the surface of cuff 102 (e.g., as seen in FIG. 1A). As the back or inside surface of the wrist W is generally planar, the rigid or semi-rigid member 114 may be similarly sized and shaped as a generally planar sheet-like member, particularly where its rigidity may prevent it from readily wrapping around the arm towards the front of the arm. In another embodiment, member 114 may be non-planar, pre-shaped with a curvature to accommodate curvature of the wrist associated with the transition from the inside of the wrist towards the opposite face of the wrist.

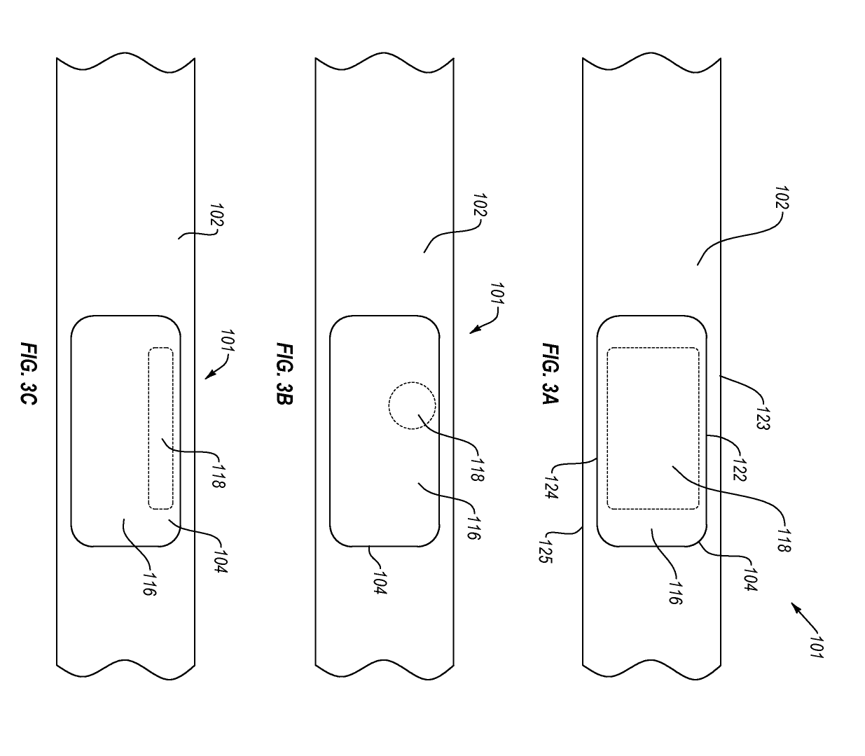

FIGS. 4A-4B illustrate an embodiment of a device 101′ including another mechanism for applying targeted compressive force to the puncture site 106. This embodiment also shows a somewhat different balloon geometry, which could be employed with any of the described embodiments. For example, FIG. 4A shows a generally circular balloon 104. For example, an outer sheath 126 may be provided for placement over balloon 104. Sheath 126 may include a hole, aperture, slit, etc. For simplicity, the term hole may be broadly employed to describe any such structure through which a portion of balloon 104 may protrude. For example, upon inflation of balloon 104, a portion 128 of balloon 104 may be received within and protrude through hole 130. Portion 128 thus protrudes and extends (e.g., towards the wrist and puncture site 106) beyond the portion of balloon 104 covered by sheath 126. Because portion 128 protrudes through hole 130, it is able to apply a focused, targeted pressure to a small localized area of the wrist W around which the cuff is placed (e.g., to radial access puncture site 106). Sheath 126 may have a thickness sufficient to provide desired comfort to the patient during application of the focused or targeted pressure to puncture site 106. Sheath 126 may be formed of a rigid or semi-rigid material (e.g., more rigid than balloon 104 and/or cuff 102), e.g., similar to member 114. Although rigid or semi-rigid member 114 is shown disposed between cuff 102 and balloon 104, it will be appreciated that such member 114 is optional. Sheath 126 may thus be rigid or semi-rigid, with sufficient thickness to limit movement under the influence of pressure applied by balloon 104.

Sheath 126 thus serves to confine and limit pressure applied by portions of balloon 104 other than protruding portion 128, in a manner similar to the embodiment described in conjunction with FIGS. 2A-3C, but without requiring that balloon 104 include a relatively thin-walled portion and a thicker-walled portion. For example, in an embodiment, sheath 126 may be separate from cuff 102, and positionable over balloon 104 during use. In another embodiment, sheath 126 may be attached to cuff 102 and/or balloon 104. Sheath 126 may be positionable over balloon 104, e.g., secured to cuff 102 and/or balloon 104, so that protruding portion 128 presses against puncture site 106 when balloon 104 is inflated.

While hole 130 is shown as generally circular, resulting in a generally circular protruding portion 128, it will be appreciated that as described relative to other embodiments, hole 130 may have any desired geometry, such as oval shaped, circular, other closed curve shape, regular or irregular polygonal shaped, other closed geometric shape, etc. Sheath 126 may similarly have any desired geometry. More than one hole 130 may be provided in sheath 126, if desired.

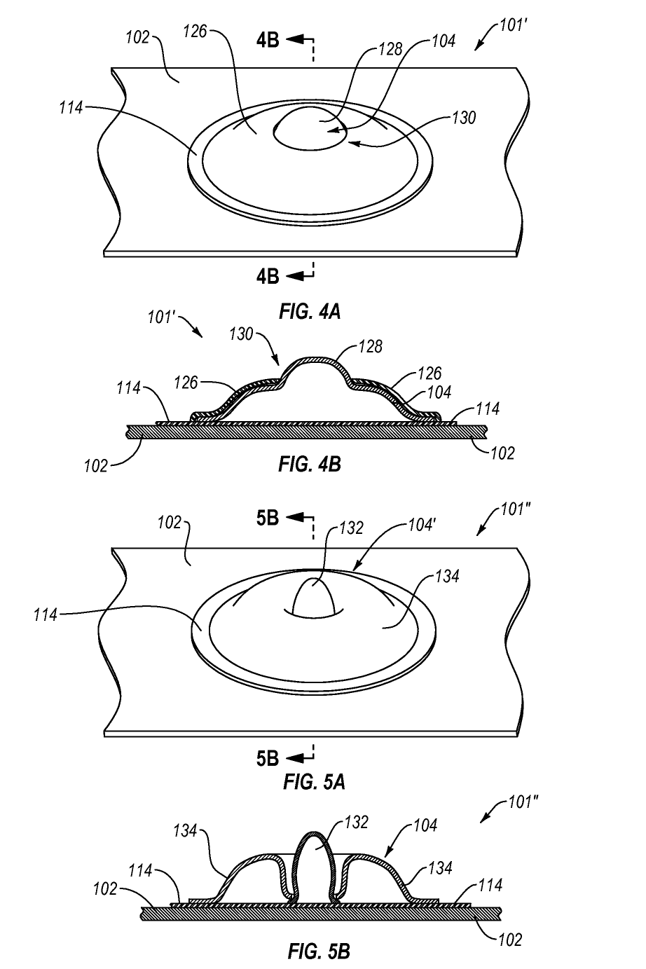

Another alternative embodiment of a device 101″ is shown in FIGS. 5A-5B, where balloon structure 104′ includes two or more balloons. A first relatively smaller balloon 132 may be provided for applying the focused, targeted pressure to puncture site 106, while a second (e.g., larger) balloon 134 may surround the first balloon, e.g., providing a geometry that may be similar or alternative to the thin-walled portions surrounded by the thicker-walled portions shown and described in conjunction with FIGS. 3A-3C. Positioning first balloon 132 over puncture site 106 results in focused, targeted application of compressive force to site 106, where smaller balloon 132 is configured to extend beyond an outer surface of balloon 134 during use (e.g., first balloon 132 extends further from cuff 102, towards wrist and radial access puncture site 106 than second balloon 134 if unconstrained). In other words, first balloon 132 stands proud relative to second balloon 134 once both balloons 132 and 134 of structure 104′ are inflated. As described above relative to the other embodiments, it will be appreciated that first balloon 132 and second balloon 134 may have any desired geometry. In an embodiment, one or both may be oval shaped, circular, other closed curve shape, regular or irregular polygonal shaped, other closed geometric shape, etc.

Another embodiment may include targeting indicia on the balloon or other portions of the cuff device, as described in further detail in a U.S. patent application bearing attorney Docket No. 16497.261, filed the same day as the present application, and herein incorporated by reference in its entirety. Such targeting indicia could be thickened, shaped or stiffened to apply focused pressure, e.g., similar to the embodiments described herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A device for applying targeted force to a radial access puncture site, the device comprising:
a cuff for placement over a wrist of a patient;
an inflatable balloon structure attached to the cuff and disposed over the radial access puncture site during use, wherein the system includes one or more features that provide for targeted application of force to the puncture site, selected from the group consisting of:
(i) wherein the inflatable balloon structure includes a balloon having a varying wall thickness so as to include a relatively thicker wall portion and a relatively thinner wall portion so that the thinner wall portion extends further from the cuff than the thicker wall portion under fluid pressure from an inflation fluid provided from a fluid source, the thinner wall portion being positioned over the radial access puncture site, such that the extended thinner wall portion of the balloon applies a focused pressure to the radial access puncture site;
(ii) an outer sheath positionable over the inflatable balloon structure during use, the outer sheath including a hole into which a portion of the inflated balloon structure is received, the portion of the inflated balloon structure protruding through the hole of the sheath during use such that the protruding portion of the inflated balloon structure applies a focused pressure to the radial access puncture site during use; and
(iii) the inflatable balloon structure including a first relatively smaller balloon disposed within a second relatively larger balloon, the first balloon being disposed over the radial access puncture site and extending beyond an outer surface of the second balloon during use, such that the first balloon applies a focused pressure to the radial access puncture site during use that is greater than pressure applied by the second balloon to areas surrounding the puncture site.

2. A system comprising the device of claim 1, further comprising a fluid source from which an inflating fluid may be delivered into the inflatable balloon associated with the cuff during use.

3. The device of claim 1, further comprising a rigid or semi-rigid member disposed between the cuff and the inflatable balloon structure to limit movement of the balloon away from the radial access puncture site when the balloon is inflated.

4. The device of claim 3, wherein the rigid or semi-rigid member extends at least to an outer peripheral edge of the balloon.

5. The device of claim 1, wherein the inflatable balloon is pre-inflated.

6. A device for applying targeted force to a radial access puncture site, the system comprising:
a cuff for placement over a wrist of a patient;
an inflatable balloon attached to the cuff and disposed over the radial access puncture site during use, wherein the balloon has a varying wall thickness so as to include a relatively thicker wall portion and a relatively thinner wall portion so that the thinner wall portion extends further from the cuff than the thicker wall portion under fluid pressure from an inflation fluid provided from a fluid source, the thinner wall portion being positioned over the radial access puncture site during use, such that the extended thinner wall portion of the balloon applies a focused pressure to the radial access puncture site.

7. The device of claim 6, further comprising a rigid or semi-rigid member disposed between the cuff and the balloon to limit movement of the balloon away from the radial access puncture site when the balloon is inflated.

8. The device of claim 7, wherein the rigid or semi-rigid member extends at least to an outer peripheral edge of the balloon.

9. The device of claim 6, wherein the thinner wall portion of the balloon is differently colored than the thicker walled portion of the balloon to mark and distinguish the thinner walled portion from the adjacent thicker walled portion.

10. The device of claim 6, wherein thinner wall portion of the balloon includes a perimeter outline or other indicia to mark and distinguish the thinner walled portion from the adjacent thicker walled portion.

11. A device for applying targeted force to a radial access puncture site, the system comprising:
a cuff for placement over a wrist of a patient;
an inflatable balloon attached to the cuff and disposed over the radial access puncture site during use;
an outer sheath positionable over the inflatable balloon during use, the outer sheath including a hole into which a portion of the inflated balloon is received, the portion of the inflated balloon protruding through the hole of the sheath during use once inflated with an inflation fluid from a fluid source such that the protruding portion of the balloon applies a focused pressure to the radial access puncture site during use.

12. The device of claim 11, further comprising a rigid or semi-rigid member disposed between the cuff and the balloon to limit movement of the balloon away from the radial access puncture site when the balloon is inflated.

13. The device of claim 12, wherein the rigid or semi-rigid member extends at least to an outer peripheral edge of the balloon.

14. A device for applying targeted force to a radial access puncture site, the system comprising:
a cuff for placement over a wrist of a patient;
an inflatable balloon structure attached to the cuff and disposed over the radial access puncture site during use, the inflatable balloon structure including a first relatively smaller balloon disposed within a second relatively larger balloon, the first balloon being disposed over the radial access puncture site and extending beyond an outer surface of the second balloon once inflated with an inflation fluid from a fluid source during use, such that the first balloon applies a focused pressure to the radial access puncture site that is greater than pressure applied by the second balloon to areas surrounding the puncture site during use.

15. The device of claim 14, further comprising a rigid or semi-rigid member disposed between the cuff and the inflatable balloon structure to limit movement of the balloons away from the radial access puncture site when the balloons are inflated.

16. The device of claim 15, wherein the rigid or semi-rigid member extends at least to an outer peripheral edge of the inflatable balloon structure.

17. A method for applying targeted force to a radial access puncture site, the method comprising:
placing a cuff of a device for applying targeted force over a wrist of a patient, the device including:
a cuff for placement over a wrist of a patient;
an inflatable balloon structure attached to the cuff that is disposed over the radial access puncture site during use, such that the inflatable balloon structure applies pressure to the radial access puncture site once inflated with an inflation fluid from a fluid source during use, the device comprising one or more features for applying a focused pressure to the radial access puncture site during use, the one or more features for applying a focused pressure comprising at least one of:
(i) the inflatable balloon structure comprising an inflatable balloon having a varying wall thickness so as to include a relatively thicker wall portion and a relatively thinner wall portion so that the thinner wall portion extends further from the cuff than the thicker wall portion under fluid pressure from a fluid source inflating the balloon, the thinner wall portion being positioned over the radial access puncture site, such that the extended thinner wall portion of the balloon applies a focused pressure to the radial access puncture site; or
(ii) the inflatable balloon structure comprising an inflatable balloon, and further comprising an outer sheath positionable over the inflatable balloon during use, the outer sheath including a hole into which a portion of the inflated balloon is received, the portion of the inflated balloon protruding through the hole of the sheath during use such that the protruding portion of the balloon applies a focused pressure to the radial access puncture site during use; or
(iii) the inflatable balloon structure including a first relatively smaller balloon disposed within a second relatively larger balloon, the first balloon being disposed over the radial access puncture site and extending beyond an outer surface of the second balloon during use, such that the first balloon applies a focused pressure to the radial access puncture site during use that is greater than pressure applied by the second balloon to areas surrounding the puncture site;
adjusting the position of one or more of the cuff, inflatable balloon structure, an optional rigid or semi-rigid member, or structure corresponding to a feature for providing focused pressure so as to align the structure for providing focused pressure over the radial access puncture site; and
inflating the inflatable balloon structure with an inflating fluid, the feature for providing focused pressure applying focused pressure to the radial access puncture site to provide hemostasis of the puncture site while limiting risk of damage to nerves adjacent the puncture site.

18. The method of claim 17, wherein the inflating fluid is cooled below ambient temperature to cool the puncture site as the inflated balloon contacts the puncture site.

19. The method of claim 17, wherein the feature for applying focused pressure comprises (i), wherein the relatively thinner wall portion extends further from the cuff than the thicker wall portion under fluid pressure from the inflating fluid, the thinner wall portion being axially aligned over the radial access puncture site, such that the extended thinner wall portion of the balloon applies a focused pressure to the radial access puncture site.

20. The method of claim 17, wherein the feature for applying focused pressure comprises (ii), wherein the portion of the balloon received within the hole of the outer sheath and protruding through the hole of the sheath is axially aligned over the radial access puncture site such that the protruding portion of the balloon applies a focused pressure to the radial access puncture site that is greater than a pressure applied by adjacent portions of the balloon.

21. The method of claim 20, the method further comprising pressing the outer sheath over the inflatable balloon while the inflatable balloon is inflated, the hole and protruding portion of the inflatable balloon being axially aligned with the radial access puncture site.

22. The method of claim 16, wherein the feature for applying focused pressure comprises (iii), wherein the first balloon is axially aligned over the radial access puncture site, the first balloon applying a focused pressure to the radial access puncture site while inflated that is greater than pressure applied by the second balloon to areas surrounding the puncture site.
